(19) United States
(12) Patent Application Publication
(10) Pub. No.: US 2008/0161844 A1
(43) Pub. Date: Jul. 3, 2008

(54) Medical Tool For Cutting a Hard Material Wall and Method of Cutting

(75) Inventor: Walter Mossle, Mittelbiberach (DE)

Correspondence Address: MARSHALL, GERSTEIN & BORUN LLP, 233 S. WACKER DRIVE, SUITE 6300, SEARS TOWER, CHICAGO, IL, 60606, US

(73) Assignee: KALTENBACH & VOIGHT GMBH, BIBERACH (DE)

(21) Appl. No.: 11/817,665

(22) PCT Filed: Mar. 3, 2006
(86) PCT No.: PCT/EP2006/001976

§ 371 Date: Sep. 27, 2007

(30) Foreign Application Priority Data
Mar. 3, 2005 (DE) 10 2005 009 802.9

(51) Int. Cl. A61B 17/32

(52) U.S. Cl. 606/171

(57) ABSTRACT

The invention relates to a medical tool for cutting a hard material wall, having a shaft which has over at least a part of its length a working region which upon vibration or rotation develops an abrasive effect. So that a handling-friendly continuous cutting of the hard material wall is achieved, there is arranged at the forward end of the shaft a flat guide sliding element projecting laterally from the shaft and intended for engagement below the hard material wall, which element is constituted smoothly at its upper side.

The invention relates to a medical tool or handpiece for cutting a hard material wall and a method of cutting a material wall using a medical handpiece with tool in accordance with the preamble of claim 1, 2 or 15.

Tools respectively mountable in a handpiece have already been proposed for the medical working of the human or animal body or artificial parts thereof, the material removing working motion of which tools is a rotation movement or a vibration movement.

A medical or dental-medical instrument and a tool connectable therewith for the material removing working of body tissue are described in DE 198 25 261 A1, whereby a plurality of tool forms are represented graphically. Among other things “sleeve-like saw blades” are described in FIGS. 7 to 9 of this document which make it possible to work a cut-out in body tissue in one working procedure, as is required e.g. for a tooth resection. Such a sleeve-like saw blade has an abrasive working surface only at its narrow side, which due to a vibration drive arranged in the associated handpiece works out the ring-like section, with material removal, only at its narrow side perimeter.

A medical or dental instrument and a tool for such an instrument are described in DE 298 10 111, whereby a forward longitudinal section of the instrument is formed by a rod-shaped handpiece with a handpiece shaft projecting forwardly, in the free end region of which a holding device for the tool is arranged and which can be brought to vibration by a vibration drive in the handpiece. In accordance with FIG. 26 of this document the tool is formed by a so-called rose drill the shaft of which is abrasive in a longitudinal section adjacent to the working head. By means of this abrasive longitudinal section of the shaft it is possible to work on the edge of an access opening of a cavity in material removing manner.

The tool or the handpiece in accordance with DE 198 25 261 A1 is configured, due to the sleeve-like form of a saw blade shaped tool, to work out a cut-out in a tissue, whereby the tool is effective at the same time abrasively over the complete perimeter of its sleeve-like form.

The tool described in the DE 298 10 114 U1 is configured to work on the edge of a cavity in body tissue, whereby the depth of the cavity is less than the depth of the body tissue. Thereby, the tool is able to work on a ring-like edge of a cavity; however, the tool is not configured to work a cut or cut-out in the body tissue.

The above-described known methods for the material removing working of the body tissue therefore are not suitable to work a separating cut or cut-out in the body tissue by means of a progressive movement of the tool in the body tissue.

For the working of a cut-out in a hard material wall, namely in the skull bone wall, it is known to drill four holes into the bone wall up to the dura (hard meninges of the brain), which forms a membrane between the skull bone and the brain, and then—starting in each case from a hole—with a keyhole saw-like working apparatus and essentially straight cuts into the bone wall, extending to the dura, progressively to work essentially straight perimeter line sections connecting the holes, until the cut-out is cut free at its perimeter. After that, a separation of the bone section from the dura is effected. This membrane may not be perforated in the above-described working of the skull bone wall. The danger of injury to the membrane is, however, very great.

The invention is based on the object of so further developing a tool or a handpiece or a method of the kind indicated in the introduction such that a handling friendly and in particular continuous cutting of the hard material wall is achieved.

This object is achieved by the features of the claim 1, 2 or 15. Advantageous further developments of the invention are indicated respectively in associated dependent claims.

The invention is based on the insight that the inner cut edge on the hard material wall is suited for forming a guide edge for the tool, so that by a sliding bearing of this guide edge the tool can be guided along the inner cut edge and the tool therefore anchored or supported and guided on the inner cut edge against a pulling out of the hard material wall.

With the medical tool according to the invention there is arranged at the end of its shaft a guide sliding element projecting to the side thereof, which is smooth at its upper side.

These above-described features of the tool are also present with the medical handpiece according to the invention.

Through this it is possible, during cutting the hard material wall, to use the guide sliding element, smooth at its upper side, as a sliding anchor part or guide part engaging behind the inner cut side edge. Due to the bearing of the smooth upper side on the inner cut side edge, caused by a slight exercise of traction, the position of the tool with respect to the hard material wall is continuously secured, so that the danger of an unintentional pulling out of the tool is avoided. Due to the abrasiveness of the working section, in functional operation of the handpiece the tool can on the narrow side be progressively moved through the hard material wall, whereby it only has to be taken care that the guide sliding element bears on the inner cut edge with its smooth upper side and slides thereon, through which the depth position of the tool is determined.

Within the scope of the invention the hole in the hard material wall, through which the guide sliding element can be inserted and placed behind the hard material wall, can be enlarged, with material removal, with the working section, or starting from the hole a slit or a cut-out can be worked with the working section, with material removal, into the hard material wall.

The invention therefore further includes the use of the medical tool for the working of a cut or cut-out in a skull bone wall.

With the invention the working of only one single starting hole in the hard material wall is required, through which in comparison with the state of the art the working of at least two further starting holes can be avoided and saved. This is made possible in that with the tool according to the invention also a curve shaped cut can be worked and therefore any desired form of the cut or cut-out can be realised. Merely one single starting hole is needed, into which the tool can be so introduced that the guide sliding element engages behind at least one of the two inner cut edges with its smooth upper side. When cutting a curve the tool is to be so guided that the guide sliding element remains behind the inner cut edge. When cutting a curve the tool is therefore to be so progressively slightly rotated that the guide sliding element is always directed transversely to the cut line. The attention of the user required for this can be reduced if the guide sliding element is formed by a flange projecting on all sides, which is arranged preferably in the form of a circle and coaxially of the shaft. With this configuration the engaging behind the hard material wall is ensured in every rotational position of the tool without the user having to pay special attention thereto.

If the tool is formed with the guide sliding element rotationally symmetrical with reference to its longitudinal middle axis, the tool is also suitable for a rotational working motion. In such a case the handpiece has a rotary drive for the tool.

The tool according to the invention, handpiece and/or method is or are suitable not only for the human or animal body but also for models or prostheses thereof, or for artificial bodies or parts of the body for the purpose of practicing the cutting or operating.

A preferred configuration for the guide sliding element is a flat limb or a flat plate, the free perimeter wall of which is rounded preferably convexly. Such a guide sliding element makes possible a gentle bearing inwardly on the hard material wall, through which injuries are avoided.

The configurations according to the invention are particularly suitable for the cutting and separating of a bone wall on the inside of which a periosteum or membrane (dura) is arranged, as is the case with the human or animal skull bone. In this case, upon cutting or separating, the guide sliding element is moved between the inner cut side edge and the membrane, whereby the membrane is lifted off from the inside of the bone wall. Through this the membrane also is handled gently, whereby the anchor and guide effect of the anchor element can be exploited. The membrane is not perforated and as far as possible also not injured. In addition, through the lifting off of the membrane the detachment thereof from the bone wall is already commenced so that the membrane can more easily be removed or released.

The method according to the invention makes possible the cutting of the hard material wall and particularly a cutting out of a cut-out with simple and friendly handling. The aforementioned advantages of the invention also can be attained.

Features are contained in further dependent claims which further simplify the medical tool or handpiece or method and make possible a rapid cutting of selected cut line forms or cut-outs.

Below, advantageous configurations and features of the invention are explained in more detail with reference to embodiments and simplified drawings. There is shown:

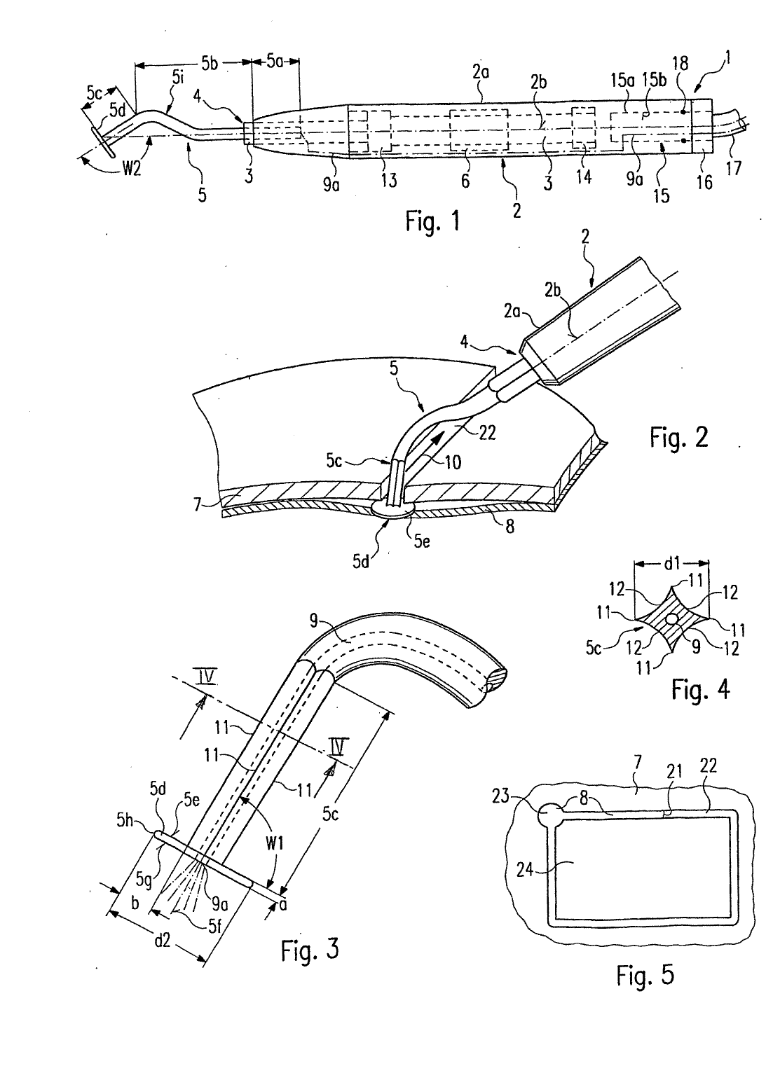

FIG. 1a medical instrument according to the invention with a handpiece and a tool attached thereto for material removing cutting of a hard material wall, in particular for cutting a bone wall, in a side view;

FIG. 2 the treatment instrument in an operating position in a perspective view;

FIG. 3 the forward end region of a tool according to the invention, in an illustration to an enlarged scale;

FIG. 4 the section IV-IV in FIG. 3;

FIG. 5 an operation site in a schematic representation.

The instrument designated as a whole by 1 in FIG. 1 is particularly suitable for the cutting and separation of a hard material wall, in particular a bone wall. The main parts of the instrument 1 are an elongate handpiece 2 with a grip part 2a which is e.g. rod-shaped and extends preferably straight, and a handpiece shaft 3 which is mounted in the grip part 2, is accessible from the forward end thereof and e.g. projects therefrom, and in the forward end region of which there is arranged a holding device 4 for a tool 5. The tool 5 is a component extending straight or angled or curved, that in its rearward end region has a connection shaft 5a for connection with the handpiece shaft 3, in its middle region has a carrying shaft 5b, in its forward end region, in which it preferably extends straight, has a working region 5c and at its forward end has guide sliding element 5d projecting to the side from the working region 5c, which in operating mode performs an anchoring and supporting function still to be described.

The working region 5c is at least at a side directed transversely to the projecting guide sliding element 5d, preferably over the complete perimeter, constituted abrasively. In connection with a vibration drive 6 arranged in the instrument 1 or handpiece, which drive is operable e.g. electrically or with compressed air, with the working region 5c, through a lateral pushing forward movement 10 of the same, a cut can be worked through a hard material wall, in particular a bone wall 7, whereby the working region 5c penetrates the hard material wall. In FIGS. 2 and 3 the hard material wall is a bone wall 7, e.g. a skull bone at which there is arranged on the inside a periosteum which below will be designated by membrane 8.

The guide sliding element 5 is, particularly at its upper side 5e, preferably on all sides, formed smooth, so that with a lateral movement relative to the hard material wall and membrane 8 is slides well and brings about only a slight sliding load.

The upper side 5e includes with the outer surface of the working region 5c or its longitudinal middle axis 5f an approximately right angle W1, which can also be slightly larger and is e.g. is approximately 90° to 110°, in particular about 95° to 100°. The axial thickness a of the anchor element 5d may be dimensioned approximately equally to the cross-sectional dimension d1 of the working region 5c or may be smaller. In the embodiment the thickness a is about 0.1 mm to 1 mm, in particular about 0.3 mm. The cross-sectional dimension d1 may be about 0.5 mm to 3 mm or more, e.g. about 1 mm to 1.5 mm. The working region of 5c can, with reference to its cross-sectional dimension d1, be formed convergently in the direction towards the guide sliding element 5.

The underside 5g extends preferably at right angles to the working region 5c or its longitudinal middle axis 5f. The free end of the anchor element 5 is, radially outwardly, preferably convergently formed, in particular rounded at its flange edge 5h, e.g. rounded in a semicircle, as FIG. 3 in particular shows. The underside 5g and/or the upper side 5e may be flat surfaces. Within the scope of the invention the guide sliding element 5d is however also advantageously useable if the upper side 5e and/or the underside 5g is or are slightly domed, e.g. in the sense of the domed shape of a discus.

The upper side 5e and/or the underside 5g can thus form supporting areas which are formed convergently towards the free end or edge of the guide sliding element, with reference to a middle plane extending at right angles to the longitudinal middle plane of the working region 5c.

The lateral overhang b, with which the guide sliding element 5c projects to the side from the working region 5, can be e.g. about 1 mm to 5 mm or more, in particular about 2 mm. Preferably the overhang b is greater than the cross-sectional dimension d1 of the working region 5c.

The flat guide sliding element 5d can project to one side or to both oppositely lying sides. In the embodiment the guide sliding element 5d is formed by a plate, which projects to all sides and is preferably formed to be circular and arranged concentrically to the longitudinal middle axis 5f, whereby its diameter d2 is about 5 mm.

The tool 5 may have a channel 9 running longitudinally in its shaft 5i, which emerges from the shaft 5i with a mouth opening 9a at such a location that a treatment medium, e.g. a cooling and/or disinfection liquid, supplied through the channel 9 is directed onto the working region 5c and/or onto the guide sliding element 5d. The channel 9 extends in the embodiment through the tool 5 up to its free end, whereby the channel 9 emerges at the underside 5g from the guide sliding element 5d approximately centrally with a jet. Instead of the treatment liquid a treatment gas, e.g. air or a mixture, can also be supplied.

The abrasiveness of the working region 5c can be realized in different ways. The outer surface of the working region 5c can e.g. be occupied with small hard particles, e.g. diamondised. In the embodiment the working region 5c has blades 11 extending approximately longitudinally, in particular straight, which may e.g. be formed by a polygonal, e.g. triangular to nonagonal cross-section. FIG. 4 shows for example a quadrilateral cross-section, in which the blades 11—formed by the corners—may have an even spacing from each other in the circumferential direction. Between the blades 1 the working region 5c may have longitudinally running hollows 12 which can preferably run out into the blades 11 and e.g. be rounded in cross-section.

The rod-like handpiece shaft 3 of the handpiece 2, projecting beyond the grip part 2a or ending approximately flush with the forward end thereof, is mounted in the grip part 2a elastically pivotable to all sides. For this there may serve elastically yielding or compressible bearing parts 13, 14, e.g. bearing rings, which are arranged at an axial spacing from one another. Due to the elastically yielding mounting of the handpiece shaft 3 this is centred in the middle, in a vibration middle position, in the rest condition.

The vibration generator or vibration drive 6 causes high-frequency short-stroke vibrations, in the sense of a vibration with a frequency preferably lying in the sonic or ultrasonic region, whereby the vibrations or amplitudes may be directed linearly, e.g. transversely and/or longitudinally of the handpiece shaft 3, or may by orbital in elliptical or circular form, in each case in a plane or, with changing of their direction, in three dimensions. Vibrations in three dimensions have proved to be particularly advantageous. Due to the radial and axial elastically yielding mounting of the handpiece shaft 3 three dimensional vibrations arise in the functional operation so that the tool 5 is abrasively effective in all directions. The respective effective direction of the tool 5, in which the latter sinks itself into the material to be worked, is directed transversely, preferably at right-angles, to the abrasive working region 5c.

In the present embodiment the vibration drive has a frequency from about 4 kHz to 8 kHz, preferably about 6 kHz, whereby in the region of the tool 5 an amplitude of the preferably three dimensional vibrations of about 0.05 mm to 0.2 mm, in particular about 0.1 mm, arises. A control apparatus can be so associated with the handpiece 2 or instrument that it makes possible a setting of the vibration performance in the aforementioned region or also a setting outside of this region so that also considerably larger amplitudes can if necessary be set.

The handpiece 2 is preferably releasably connected to a connection piece 16 by means of a quick-fastening coupling 15, in particular a screw coupling or a plug-in coupling, from which connection piece a supply line 17 extends to a non-illustrated supply unit.

The plug-in coupling is preferably such a coupling that in the coupled condition a rotation of the handpiece 2 around its longitudinal middle axis 2b, and thereby a passage of the at least one treatment or cooling medium in a media line 9a, is ensured, which media line extends forwardly through the handpiece 2 up to the tool 5, where it is connected to the channel 9. Such a coupling, designated as a plug-in/turn coupling, is formed with a cylindrical or stepped-cylindrical coupling pin 15a and a coupling recess 15b rotatably accommodating the coupling pin, whereby in the present exemplary configuration the coupling pin 15a projects forwardly from the connection piece 16 and the coupling recess 15 opens out to the rear from the handpiece 2. In the coupled condition, an unintended release of the plug-in/turn coupling is prevented by a releasable, elastic securing device 18 known per se, which can in particular be manually overcome. For a coupling or separating process elastic securing device 18, effective with an elastic biased securing element, can be overcome and latched or released in a handling-friendly manner.

With the instrument 1 or handpiece 2 according to the invention and the tool 5, in a simple and advantageous as well as handling-friendly way, a hard material wall can be worked in a narrow-side material removing manner or separated by cutting or a cut-out 21 cut out of the hard material wall. In these cutting processes the tool 5 is applied on the narrow side to the hard material wall so that the guide sliding element 5 engages under or behind the hard material wall. The tool is then, with its working region 5c, with the drive 6 switched on, so moved laterally against the hard material wall 7 that the working region 5c is effective abrasively or in material removing manner and mills or works a slit 22 into the material wall.

It is advantageous for reasons of ergonomic handling if the working region 5c extends at an angle to a connection shaft 5a of the tool 5 or to the longitudinal middle axis 2b of the handpiece 2, whereby the angle W2 included therebetween is obtuse and is preferably approximately 105° to 135°, in particular 120°. Thereby the working section 5c and a section of the shaft 5i turned theretowards may be angled or curve shaped to the side away from the guide sliding element 5d.

In the embodiment, the guide sliding element 5d is formed by a flat plate which preferably projects to all side from the working region 5c. Within the scope of the invention the guide sliding element 5d can be formed by flat limb projecting to the side. Also in the case of such a configuration of the anchor element 5d this is able to ensure the guiding and supporting function desired in the operating mode, in which the guide sliding element 5 is guided on the underside of the bone wall. Thereby, the operating person does not need to pay attention to the tool 5 slipping out from the slit 22, since this is prevented by the guide sliding element 5 through bearing on the underside.

If the guide sliding element 5d is formed only by a lateral limb, the pushing forward movement 10 is to be carried out transversely of this limb, to ensure the aforementioned guiding and supporting function. Within the scope of the invention a flat limb forming the guide sliding element 5d can also project to both sides, so that both slit edges are engaged beneath.

When a cut-out 21 of selected form, e.g. a quadrilateral cut-out 21 in accordance with FIG. 5, is to be worked into the hard material wall, a hole 23 is worked into the hard material wall in the marginal area of the desired cut-out 21, e.g. drilled in, which is so large that the forward end of the tool 5, with the guide sliding element 5d projecting at the side, can be introduced therein into an above-described starting position, in which the cutting process can begin.

The configuration according to the invention and the method according to the invention are particularly advantageously suitable for cutting a hard material wall, in particular a bone wall 7 on the inside of which a membrane bears, which in the case of a bone wall 7 is formed by a periosteum. In the case of a skull bone this is the dura. In the case of a sinus lifting in the maxillary sinus this is e.g. Schneider's membrane. Such a type of membrane protects the tissue lying thereunder and may not be perforated; on the contrary, a gentle treatment is desired.

Also a bone wall 7 occupied on the underside with a membrane 8 can be worked, cut or cut-out with the tool 5 or handpiece 2 according to the invention on the narrow side, as has already been described, without the membrane 8 being perforated or injured. It is thereby significant that the tool 5 is so applied that the guide sliding element 5d reaches between the membrane 8 and the bone wall 7 and is there guided. This can be achieved in simple manner in that the tool 5, upon maintenance of a slight pressure of the anchor element 5d against the underside of the bone wall 7 moves with narrow side against the bone wall 7 and is pushed, see pushing forward direction 10.

After a cutting of a cut-out 21 in the above-described sense and return of the working region 5c to the hole 23 or another hole, the so formed bone section 24 can be released from the membrane 8 and removed. After that, the operation site is available for an operation below the bone wall 7.

If the tool 5 with the guide sliding element 5c is constituted rotationally symmetrically with reference to its longitudinal middle axis, it is also suitable for a rotating working motion. In such a case the handpiece 2 has a rotary drive for the tool 5.

1. Medical tool for cutting a hard material wall, said tool comprising a shaft defining a working region over at least a part of its length, which working region develops an abrasive effect upon vibration or rotation thereof, and a flat guide sliding element fixed and projecting laterally from a forward end of the shaft for engagement below the hard material wall, said element arfining a smoothly upper side.

2. In combination, a medical handpiece and a tool according to claim 1, said handpiece having a drive for vibrating or rotating the tool.

3. Tool according to claim 1 wherein at least one of the upper side an underside of the guide sliding element is formed by a flat supporting surface or by a support surface convergent toward a free end of the guide sliding element.

4. Tool according to claim 1, wherein the upper side includes an angle of about 90° to 110° with a longitudinal middle axis of the working region.

5. Tool according to claim 1,
wherein a thickness of the guide sliding element is less than the cross-sectional size of the working region.

6. Tool according to claim 1,
wherein a free edge of the guide sliding element is constituted convergently.

7. Tool according to claim 1, wherein the guide sliding element projects from its working region to at least two oppositely lying sides, preferably on all sides.

8. Tool according to claim 1, wherein the guide sliding element has a round peripheral form.

9. Tool according to claim 1, wherein the working region has at a perimeter a plurality of blades.

10. Tool according to claim 9, wherein the blades are formed by a polygonal cross-sectional form of the working region.

11. Tool according to claim 1, wherein a cross-sectional size of the working region is about 0.5 mm to 3 mm.

12. Tool according to claim 1, wherein the working region extends transversely with reference to a longitudinal axis of a connecting shaft of the tool.

13. Tool according to claim 12, wherein the working region of the tool includes with the connecting shaft an obtuse angle which is about 95° to 145°.

14. Tool according to claim 1, wherein a channel extends forwardly in the shaft of the tool, which channel emerges from the tool at an opening which is directed onto the working region or onto the guide sliding element, or emerges at the underside of the guide sliding element (5d).

15. Tool according to claim 4 wherein said angle is about 90° to about 110°.

16. Tool according to claim 4 wherein said angle is about 105°.

17. Tool according to claim 5 wherein said cross-sectional size is about 0.1 mm to about 1 mm.

18. Tool according to claim 5 wherein said cross-sectional size is about 0.3 mm.

19. Tool according to claim 6 wherein said free edge is rounded.

20. Tool according to claim 7 wherein the guide sliding element projects from the working region on all sides.

21. Tool according to claim 9 wherein said blades extend in the longitudinal direction of the working region.

22. Tool according to claim 10 wherein said polygonal cross-sectional form is selected from the group consisting of triangular to nonagonal forms.

23. Tool according to claim 11 wherein said cross-sectional size is about 1 mm to about 1.5 mm.

24. Tool according to claim 13 wherein said obtuse angle is about 120°.
